(19) United States
(12) Patent Application Publication
(10) Pub. No.: US 2023/0270985 A1
(43) Pub. Date: Aug. 31, 2023

(54) MICRONEEDLE DELIVERY DEVICE

(71) Applicant: University College Cardiff Consultants Limited, Cardiff South Glamorgan (GB)

(72) Inventors: James Birchall, Cardiff South Glamorgan (GB); Sion Coulman, Cardiff South Glamorgan (GB); Benedetta Gualeni, Cardiff South Glamorgan (GB); Oliver Bishop, Petworth West Sussex (GB); Nicholas Smart, Petworth West Sussex (GB); Isabelle Stauber, Petworth West Sussex (GB)

(73) Assignee: University College Cardiff Consultants Limited, Cardiff South Glamorgan (GB)

(21) Appl. No.: 18/015,315

(22) PCT Filed: Jul. 1, 2021
(86) PCT No.: PCT/GB2021/051672

§ 371 Date: Jan. 9, 2023

(30) Foreign Application Priority Data
Jul. 10, 2020 (GB) 2010674.6

(51) Int. Cl. A61M 37/00

(52) U.S. Cl. CPC A61M 37/0015; A61M 2037/0046; A61M 2037/0061; A61M 2037/0023

(57) ABSTRACT

The invention concerns a device for introducing microneedles into the skin of a subject; and a method for inserting microneedles into the skin of a subject comprising use of said device.

### FIELD OF THE INVENTION

The invention concerns a device for introducing microneedles into the skin of a subject; and a method for inserting microneedles into the skin of a subject comprising use of said device.

### BACKGROUND OF THE INVENTION

Biopharmaceuticals are delivered predominantly using hypodermic needles, with over 16 billion injections given worldwide each year. However, injections using hypodermic needles are not generally well-accepted by patients due to pain, bleeding, fear of needles, and the need for administration by skilled healthcare workers. As an alternative, microneedles (MNs) have been investigated for a diversity of medical applications, especially where the delivery of bioactives to the skin is the primary focus.

MNs are micron-sized, needle-like projections, often organized in an array having a defined geometric pattern on a planar base plate and, amongst other applications, are currently being exploited for the targeted intraepidermal and intradermal delivery of drugs and vaccines. Due to their microscopic dimensions MNs do not penetrate skin deep enough to cause any significant pain, bleeding or scarring, as demonstrated through numerous clinical trials. The application of MNs to the skin surface results in penetration of the outer skin barrier, the stratum corneum (SC) without impinging significantly on nerves or blood vessels. Most notably, the micron-scale dimensions of the MNs allow for their simple and direct application into skin without requiring professional training, and therefore they avoid the shortcomings of conventional hypodermic needles.

MN fabrication has dramatically grown over the past 15 years. This technology has enabled a variety of different MNs to be made for drug delivery to the skin and other targets. MNs have been fabricated out of many different materials including silicon, metals, polymers, and ceramics using a variety of different fabrication methods including lithography, wet and dry etching, laser cutting and micro-moulding. This has led to various types of MNs being developed, mainly: (i) solid microneedles for skin pre-treatment to increase skin permeability, (ii) microneedles coated with drug that dissolves and run off in the skin, (iii) dissolving polymer microneedles that contain drug and both the microneedle and the drug fully dissolve in the skin and (iv) hollow microneedles for drug infusion into the skin.

Dissolving or degrading MNs are commonly made of a biodegradable polymer that contains pharmaceuticals within a polymer matrix, wherein the pharmaceutical is released after skin insertion/embedding via dissolving or degradation of the polymeric matrix. Dissolving or degrading MNs have received attention as an innovative transdermal drug delivery system due to minimum pain on delivery, biocompatibility and patient convenience. However, in spite of these advantages, some challenges for the complete delivery of such pharmaceuticals exist because of the incomplete or irreproducible insertion of the dissolvable MNs. For example, due to the viscoelastic properties of the skin, dissolvable MNs may lack the requisite rigidity, compared to their solid material counterparts, to completely penetrate the skin surface and so often fail. Further, even if capable of penetrating the skin, often the complete delivery of the pharmaceutical agent is not achieved.

The successful use of MNs often depends on the function of the device used for MN insertion into the skin, skin recovery, and drug stability during manufacture, storage and delivery, and also on patient outcomes, including lack of pain, skin irritation and skin infection. Usually, dissolvable or degradable MNs are fabricated on an adhesive patch that facilitates their insertion into the skin and aims to keep them on or in the skin until they are completely dissolved, however, this arrangement can be associated with allergic responses and has been reported to be uncomfortable. Further, the patch must be in intimate contact with the skin for the time necessary to deliver the pharmaceutical agent which, in the case of long-term agent delivery, can be problematical. Additionally, in situations requiring long-term agent delivery, it is essential that the microneedles are fully inserted and firmly embedded into the skin in order to ensure correct dosing.

Consequently, we herein describe a novel device for the simple introduction and embedding/implantation of microneedles into the skin for use, for example, in intradermal drug delivery and with particular application in the context of long-term agent delivery. Notably, the device is configured to prevent exposure of microneedles during transport and storage, and to control the force of insertion of the microneedles into the skin. Further, and importantly, the device is also configured so that insertion and detachment of the microneedles, and so proper embedding in the skin, is easily achieved, this allows for consistent and reproducible implantation of MNs, which ensures reliable delivery of agents, such as therapeutics and pharmaceuticals, at the correct dosage and for prolonged periods of time.

### Statements of Invention

According to a first aspect of the invention there is provided a device [1] for introducing/embedding microneedles into the skin of a subject said device comprising:
a first part [2] comprising at least one carrier [3] having integral or associated therewith a plurality of microneedles [4] wherein said carrier

is moveably mounted in a first housing [2a] to move with respect to said housing [2a] from a retracted to an extended position where, when in said extended position, said microneedles or part thereof [4] project beyond said housing [2a] and further wherein said at least one carrier

is also moveably mounted in said housing [2a] to move with respect to said housing [2a] laterally or at 90° with respect to said movement that produces the afore extension;
a second part [5] comprising at least one actuator [6] which is mounted in, or integral with, a second housing [5a] that is adapted to engage with said first housing [2a], wherein said actuator [6] and/or said second housing [5a] is movable with respect to said first housing [2a] so that said actuator [6] is functionally coupled with said at least one carrier [3] whereby, when the actuator [6] is activated:
   i) the actuator [6] exerts a force to move said carrier [3] with respect to said first housing [2a] from said retracted to said extended position and, when in said extended position,
   ii) said actuator exerts a force to move on said carrier [3] with respect to said first housing [2a] laterally or at 90° with respect to said movement that produces the afore extension, thereby breaking the microneedles to leave parts of same in the skin.

In a preferred embodiment of the invention said carrier [3] is mounted on or in a plate [7] or between a pair of plates [7]. Plate(s) [7] is/are, ideally, adapted to engage a movable support [8] that can move with respect to said first housing [2a] from a retracted to an extended position where, when in said extended position, said microneedles [4] or part thereof project beyond said first housing [2a] and further wherein said carrier [3] or plate [7] is also moveably mounted in said first housing [2a] to move with respect to said first housing [2a] laterally, or at 90° with respect to said movement that produces the afore extension. In an alternative embodiment of the invention, said carrier [3] is mounted directly on said movable support [8] which performs the afore functions.

Most preferably a plurality of said carriers [3] are provided and each is, ideally, mounted on one, or between a pair, of said plates [7]. More ideally still a plurality of said plates [7] are mounted on said movable support [8] in an aligned manner. More ideally still movement of said movable support [8] brings about synchronised and ideally coordinated movement of said plates [7] whereby said plates [7] move together from a retracted to an extended position, either simultaneously or in a synchronised fashion. In an alternative embodiment of the invention, said carrier [3] is mounted directly on said movable support [8] which performs the afore functions.

In a further preferred embodiment, a plurality of said movable supports [8] are provided, each carrying a plurality of said plates [7] whereby a number of plates [7] are housed within said first housing [2a], ideally, to form at least one array [9]. In an alternative embodiment of the invention, a plurality of said carriers [3] are mounted directly on said movable supports [8] whereby a number of said carriers are housed within said first housing [2a], ideally to form at least one array [9].

More ideally still said first [2a] and second housings [5a] are movably engaged there together whereby they can move with respect to each other.

Preferably said at least one actuator [6] is a fixed part of said second housing [5a] but able to move with respect to said first housing [2a] by way of movement of said first [2a] and second housings [5a] with respect to each other, ideally manually operated movement.

More preferably still, said plurality of carriers [3] or plates [7] are mounted on said movable support [8] and, under the influence of said actuator (6), said support [8], by way of a downward stroke, moves said carriers [3] or plates [7] from a retracted to an extended position where, when is said extended position, said MNs [4] or part thereof project beyond said first housing [2a].

Yet more preferably, said plurality of carriers [3] or plates [7] are mounted on said movable support [8] to slide laterally whereby once, under the influence of said actuator [6], said support [8], by way of a downward stroke, moves said carriers [3] or plates [7] from a retracted to an extended position, when in said extended position, the continued downward stroke of said actuator [6] is translated into a lateral force as a result of a stopping member [10] preventing further downward motion, this lateral force then forces said carriers [3] or plates [7] there apart whereby said carriers [3] or plates [7] move laterally, and ideally, a number of carriers [3] or plates [7] move laterally in a first direction and another number move laterally in a second opposite direction, with respect to said first direction. Most preferably about an equal number of carriers [3] or plates [7] move in said lateral first and second directions.

Yet more preferably still, at least one of said housings [2a, 5a] comprises an indicator [11] which indicates when the extended/downward and/or lateral movement is complete. Preferably, said indicator [11] is a marker that becomes visible, or sits in a more visible position, once said afore movement(s) is/are complete to indicate that said microneedles [4] have penetrated and/or been left in the skin.

In a yet further preferred embodiment of the invention said actuator [6] makes contact with said movable support [8] and by doing so moves said carriers [3] or plates [7] mounted on same. Preferably a number of carriers [3] or plates [7] are provided each one mounted on the same or different movable supports [8].

Reference herein to a microneedle(s) [4] refers to at least one microneedle, ideally a plurality, wherein said microneedle(s) have the requisite length, diameter and physical arrangement necessary for insertion into the skin. In a preferred embodiment, the microneedle(s) have a length of from about 10 μm to about 1500 μm. More preferably, the microneedle(s) have a length of from about 50 μm to about 1000 μm.

More preferably still, said microneedle(s) comprise a point of weakness to facilitate breakage such as, but not limited to, a taper and further include a neck region [4a] or indented region which forms a point of weakness for the breakage of same upon lateral movement and so helps with detachment of said MNs from the carrier so that the MNs, or at least their tips, are deployed within the skin. In this embodiment, it advantageously has been found that the detached microneedles exhibit improved anchorage in the skin thus facilitating skin embedding/insertion. Preferably, said points of weakness are manufactured towards or at the base of the, or each, microneedle such that the majority of said microneedles are delivered into the skin.

Preferably said microneedles [4] are circular or semi-circular in cross section and when the latter they are selectively aligned so that, in use, their curved surface faces towards the downward stroke of the actuator [6] and their flat surface away from the downward stroke of said actuator [6], or vice versa.

In a further preferred embodiment, said microneedles [4] are dissolvable or degradable or substantially dissolvable or degradable microneedles. Dissolvable or degradable microneedles are known in the art and refer to those microneedles that are manufactured from a solid, or substantially solid, material but wherein said solid material has the ability to pass or reduce into the surrounding environment under certain conditions, such as when in contact with an aqueous environment or the skin. Ideally the microneedles are made from a polymer which can be bio-absorbable or biodegradable. In a preferred embodiment, said dissolvable microneedles may be manufactured from materials of synthetic or natural origin. Non-limiting examples of suitable materials include: vinyl polymers such as polyvinyl alcohol (PVA), polyvinyl chloride, polyvinyl fluoride, polystyrenes, poly(vinyl imidazole) and polymers of ethylene-vinyl acetates; polyacrylates; polyurethanes; polyesters such as poly(valeric acid), poly butyric acid, poly lactides (PLA), polyglycolides (PGA), poly (lactide-co-glycolide) (PLGA), poly caprolactone (PCL), poly butylene succinate (PBS), poly p-dioxanone (PPDO), and aromatic co-polyesters; polyethylene oxide; chlorosulphonate polyolefins; polyanhydrides; polyorthoesters; polysaccharides such as acyl substituted cellulose acetates; polycarbonates; polyamides and poly ester-amides. Mixtures and/or copolymers of the above materials may also be used.

In a preferred embodiment, said dissolvable or degradable microneedles [4] and said carrier [3] are manufactured from the same material.

Alternatively, said dissolvable or degradable microneedles [4] and said carrier [3] are manufactured from different materials, for example where the cost of agent to be delivered is substantial, the microneedles are made from a material that contains or is impregnated with said agent and the carrier is made from any suitable material known in the art that can function as a support or substrate for the microneedles such as, but not limited to, metals, ceramics, organics, polymers or composites or the like, so minimizing material/agent waste. Further, as will be appreciated, through use of different polymers for the microneedle and the carrier, differential mechanical properties can be achieved thus facilitating MN detachment from the carrier.

Alternatively, said microneedles are not necessarily dissolvable or degradable and so are coated with a delivery agent.

Accordingly, in a preferred embodiment of the invention, said microneedles [4] comprise at least one therapeutic, pharmaceutical, cosmetic or biological agent for delivery into the skin. Said agent(s) include(s) active agents intended for topical, local, and/or systemic delivery. Generally, any drug or active agent can be delivered using the microneedles of the present invention, specifically those that are known to be effective when delivered via the skin. Preferably, said agent includes any conventional medicament, therapeutic, contraceptive or cosmetic agent, vaccine, protein, antibody, or structural agent.

In a preferred embodiment said microneedles [4] are either hollow or solid. When the microneedles are hollow, said agent(s) is/are ideally loaded into the hollow portion of the MNs and delivered to the subject on insertion of the MNs into the skin and/or as the MNs dissolve. When the MNs are solid, said agent(s) is/are loaded in the material of the MNs, and delivered to the subject on insertion of the MNs into the skin and/or as the MNs dissolve.

Depending upon the agent(s) to be delivered, as well as the desired length of time of delivery, and the polymer used to form the MNs, the MNs can be configured to dissolve at a predetermined rate to thus release said agent(s) at a predetermined rate when embedded in the skin.

Thus, MNs [4] which are detached from the device and left embedded in the skin can provide for sustained or extended release of agent(s) delivered by the MNs [4].

In a preferred embodiment, said microneedles are configured for rapid release of said agent(s) and thus they are made from a material that rapidly dissolves in the skin. Alternatively, said microneedles are configured for prolonged and sustained release of said agent(s) and thus they are made from a material that slowly dissolves in the skin. Reference herein to rapidly dissolving MNs is to needles that delivers their agent(s) within minutes, hours or a day of insertion and reference herein to slowly dissolving MNs is reference to needles that delivers their agent(s) within days or months or years of insertion.

In a particularly preferred embodiment of the invention, said first [2a] and second housings [5a] are movably engaged there together whereby they can move with respect to each other and said actuator [6] is a fixed part of said second housing [5a]. In use, such a device [1] is placed against a site where microneedles are to be embedded/inserted and movement of said second housing [5a], by the depression of same, generates a downward stroke which, via said actuator [6], moves said carriers [3] or plates [7] mounted on said movable support [8], to an extended position whereby said microneedles or parts thereof [4] project beyond said first housing [2a] (into skin). Moreover, continued downward movement of said second housing [5a] is translated, by a stopping member [10] that prevents further downward movement, into a lateral force that slides said carriers [3] or plates [7] laterally along the movable support [8], resulting in a shearing force being exerted on said microneedles [4] which thus break at their point of weakness. At this point the microneedles [4] are detached from said housing [2a] and remain in the skin. Thus, a single downward depression of one of the two housings [5a] results in microneedles [4] being extended into adjacent skin and then sheared away from the housing [2a]. Further, and notably, the device [1] has been designed with the force of the downward stroke ensuring rapid but comfortable insertion of said MNs into the skin each time the device is used thus ensuring the device provides a reliable and reproducible experience. Ideally, MN insertion is completed within about one second (the time it takes to activate the device) and therefore the process is designed to be quick, effective and easy for the user.

In yet a further preferred embodiment, the device comprises a removable shield or cap [12] adapted to prevent movement of the first part [2] or first housing [2a] with respect to said second part [5] or second housing [5a], for example, during transport.

According to a second aspect of the invention, there is provided a method for inserting microneedles into the skin of a subject, said method comprising the steps of:
a) applying to the skin of said subject a device [1] as described herein;
b) activating the actuator [6] of the device to move the at least one carrier [3] or at least one plate [7] with respect to said first housing [2a] from a retracted to an extended position to insert the microneedles [4] into the skin;
c) further activating or continuing activation of said actuator so that said actuator [6] exerts a force on said at least one carrier [3] or at least one plate [7] to move it/them with respect to said first housing [2a] laterally, or at 90° with respect to said movement that produces the afore extension, to detach the microneedles [4] from the carrier [3] or plate [7 ]; and
d) removing the device [1] from the skin.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

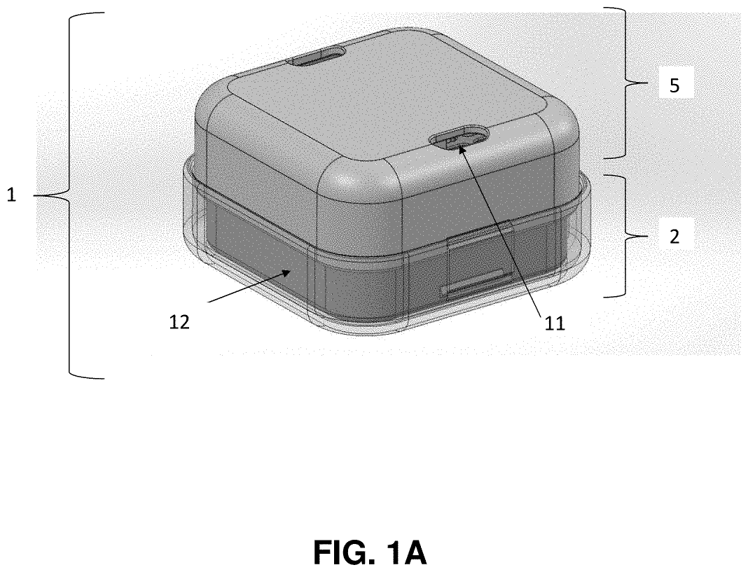

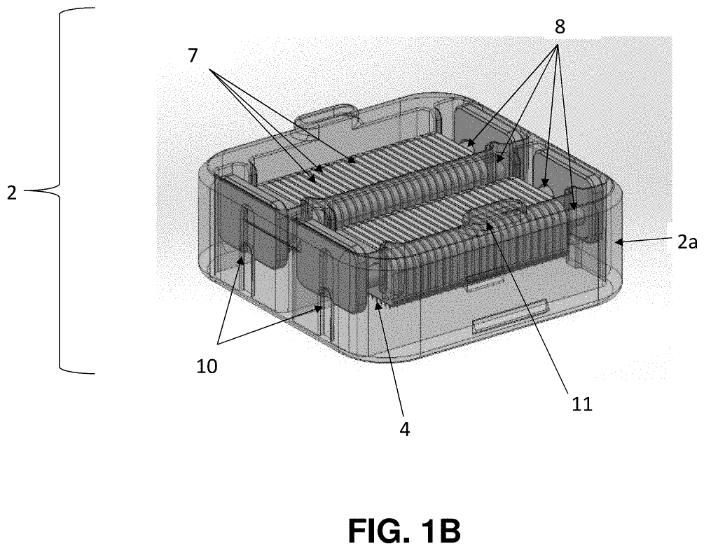

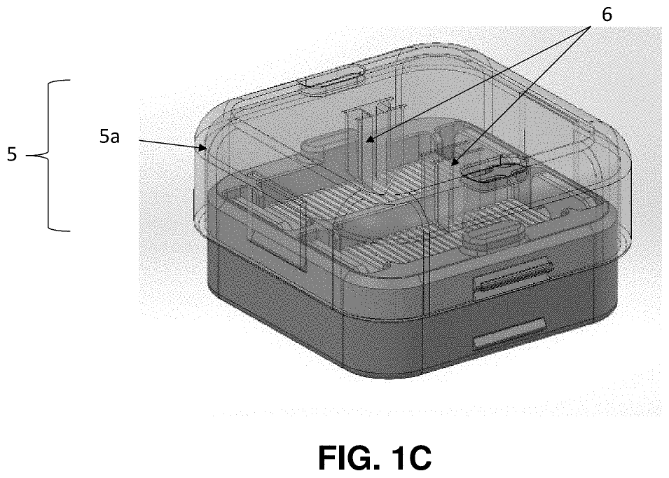

FIG. 1A shows a perspective view of the device in accordance with the embodiment of the invention when fully assembled;

FIG. 1B shows a perspective view of the first part of the device according to an embodiment of the invention comprising two microneedle arrays supported within the housing, each array being formed by a plurality of microneedle carrier plates aligned on moveable supports;

FIG. 1C shows a perspective view of the device according to FIG. 1A with a transparent housing of the second part of the device showing the actuators associated therewith according to a preferred embodiment;

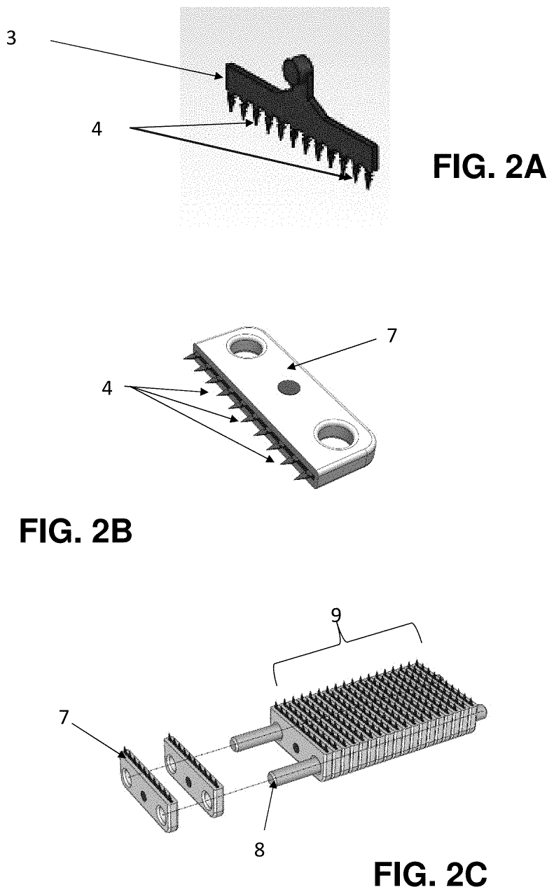

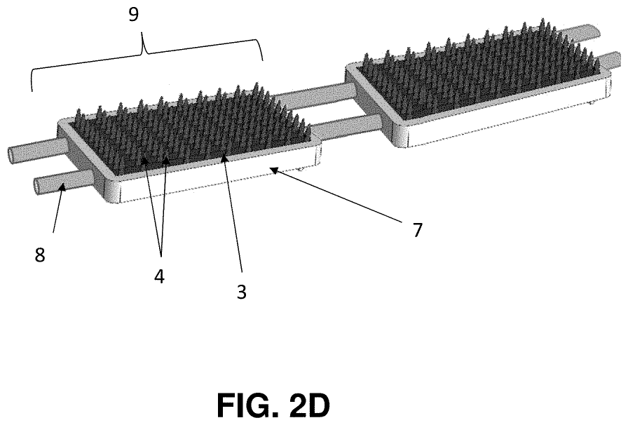

FIG. 2A shows a perspective view of a carrier according to an embodiment of the invention comprising a plurality of microneedles;

FIG. 2B shows a perspective view of a single plate according to an embodiment of the invention comprising the microneedle carrier shown in FIG. 2A;

FIG. 2C shows a perspective view of a plurality of the plates shown in FIG. 2B arranged in an aligned manner on two movable supports to provide a microneedle array;

FIG. 2D shows a perspective view of a carrier adapted to provide a microneedle array according to an alternative embodiment of the invention where two carriers are mounted in separate, single plates that are supported using two, spaced movable supports;

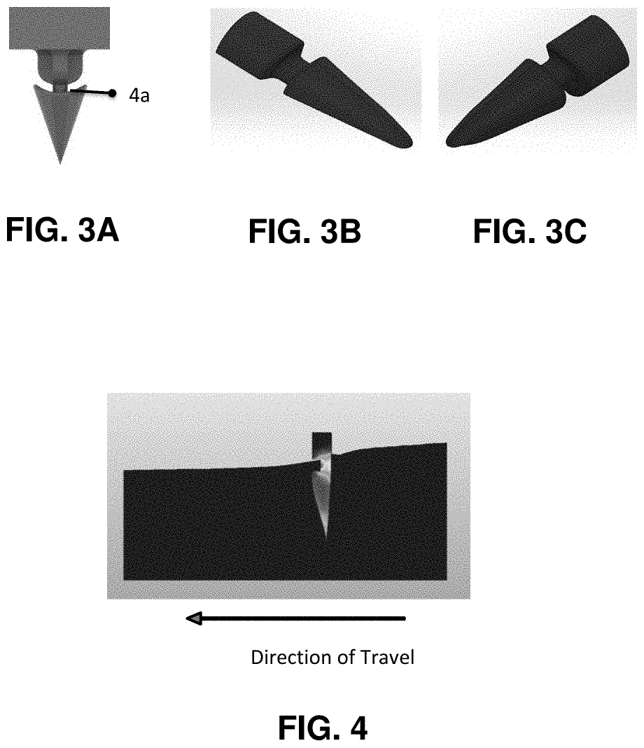

FIG. 3A shows a side sectional view of an exemplar microneedle used in the device according to an embodiment of the invention;

FIG. 3B shows an upper perspective view of an exemplar microneedle used in the device according to an embodiment of the invention;

FIG. 3C shows a lower perspective view of an exemplar microneedle used in the device according to an embodiment of the invention;

FIG. 4 shows a cross-sectional side view of a microneedle according to an embodiment of the invention when inserted into a simulated skin model and showing (using shading) the forces exerted on the needle are concentrated at the microneedle neck where a point of weakness is provided to facilitate needle breakage under stress brought about by lateral movement; and

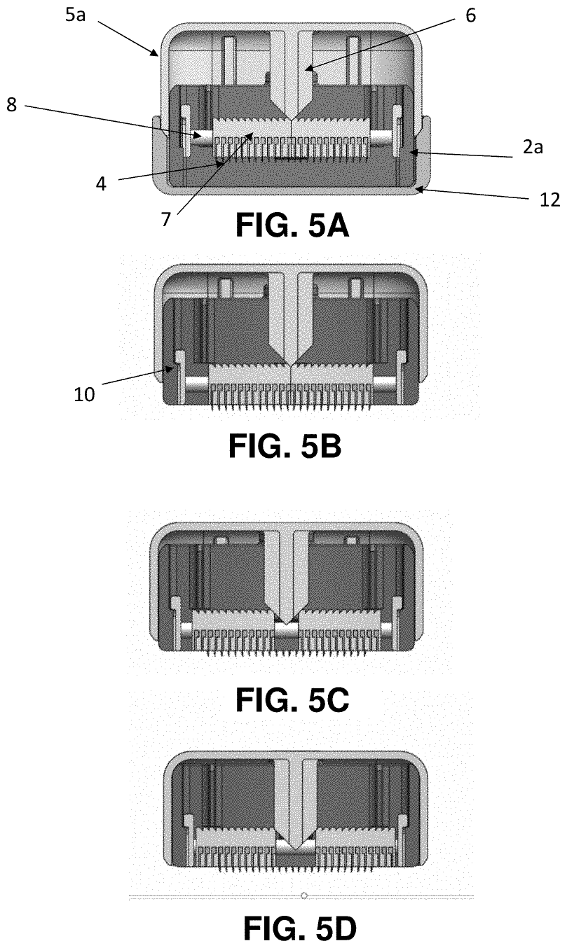

FIG. 5 shows a cross-sectional side view of the device of FIGS. 1-2 during use.

Referring now to the figures and, firstly, to FIG. 1A there is shown a perspective view of a typical device [1] of the invention. Device [1] comprises a lower first [2] and an upper second part [5] adapted to be moveable with respect to one another and each comprising a housing [2a & 5a]. Although the first part [2] and second [5] part is illustrated as a single unit, those skilled in the art will appreciate that they are provided as two parts adapted for relative movement.

For convenience the device is shown as having a square cross-section although alternative shapes may be made, according to a user's requirements.

The internal components of the parts [2] and [5] are best described by way of the subsequent figures.

In FIG. 1B, there is shown a perspective view of the first part [2] of the device comprising a first housing [2a] that houses internal components including at least one, and more ideally, a number of plates [7] each having associated therewith, or attached thereto in conventional manner, a carrier [3] fashioned to provide a plurality of microneedles [4].

The carriers [3] are best shown according to the embodiment depicted in FIG. 2. As shown in the perspective views of FIGS. 2A and 2B, the microneedles [4] are provided in a row on a carrier [3]. Carrier [3] is mounted on a plate [7] that is adapted, by way of holes, to engage or sit on an elongate movable support [8] within, and moveable with respect of, the first housing [2a]. Notably the holes are such that the plates can slide along movable supports [8]. Alternatively, as shown in FIG. 2D, at least one or more carriers are provided in the form of a substrate providing a plurality of microneedles [4] to form an array [9] and said substrate is mounted on at least one elongate movable support [8].

With reference to the embodiment shown in FIG. 1B and FIG. 2C, carriers [3] are mounted on (or in or between) plates [7] and a plurality of such plates are provided in an aligned fashion to form a microneedle array [9] mounted on movable supports [8]. The device of the invention is shown comprising two arrays [9], but as will be appreciated, more or less can be included according to the number of microneedles required to be inserted into the skin, the number of movable supports [8] and depending on the intended use. Further movable supports [8] may be arranged within housing [2a] in any fashion.

With reference to the embodiment shown in FIG. 2D, carriers [3] provide at least two separable substrates mounted on (or in or between) plates [7] and providing a plurality of microneedles to form a microneedle array [9] mounted on at least one, and in this embodiment two, elongate movable supports [8].

With reference to FIG. 1C, it can be seen that housing [5a] on its inner side includes at least one, and in this embodiment, a pair of actuators [6], or downwardly depending mating members, that can be moved to a position where they engage with plates [7]. Ideally the contact part of actuators [6] is fashioned into a tip thus providing a point through which force is exerted.

Notably, the carriers [3], or plates [7] when included, are movably mounted on supports [8] to move down with respect to housing [2a], via the contact of actuators [6] with plates [7].

Further, at least one carrier [3] or plate [7] is also moveably mounted on a support [8] to move laterally along support [8]. Thus, the carriers [3] or plates [7], can move downwardly on support [8] and slide laterally along support [8].

As will be appreciated, the number and shape of the moveable supports [8] can vary provided that both downward and lateral movement is achieved which, as will become apparent, is important to effect penetration of the microneedles and the detachment of the microneedles from the housing [2a].

The device [1] and more specifically the first housing [2a], is provided with a dampener or stopping member [10] which acts to limit the extent of downward movement of the moveable support [8] (and so carriers [3] and/or plates [7] of the microneedle array [9]) and thereby limit the extending of the microneedles [4] from housing [2a] and so the depth of penetration into skin. Thus, the location of stopping member [10] with respect to movable support [8] is chosen to ensure the correct depth of extension and so insertion of the microneedles [4]. Further, the said location of stopping member [10] affects the downward force so that carrier and/or plate movement cannot be so great as to break the microneedles [4] during injection or penetration into the skin. Conventional stopping mechanisms such as aligned runners on the moveable support and/or housing, or other stops may be used for this purpose and are known to those skilled in the art.

Moreover, and importantly, by controlling downward extension in this fashion, in particular stopping the downward motion before completion of the downstroke of actuators [6], enables the last part of the downward stroke to be converted into a lateral force which forces said carriers [3] or plates [7] apart whereby, typically, all of said carriers [3] or plates [7] move laterally.

Thus, a first part of the downward stroke extends carriers [3] or plates [7] outside housing [2a] and so into adjacent skin and a second part of the downward stroke moves the carrier [3] or plates [7] laterally, effectively snapping the microneedles [4] and bringing about detachment of same from the device [1].

Ideally, a number of carriers [3] or plates [7] move laterally in a first direction and another number move laterally in an opposite direction, with respect to said first direction, typically half move to the left and half move to the right. In this way, it has been found that lateral force can be exerted upon the needles [4] to ensure breakage.

Notably, where the carriers [3] or plates [7] are arranged in an aligned manner, movement of more than one movable support [8] brings about synchronised and ideally coordinated movement of said carriers [3] or plates [7] whereby said carriers [3] or plates [7] move simultaneously together from a retracted to an extended position.

As shown in FIG. 1A-C, the device is provided with an indicator [11] that signals when the extended/downward and/or lateral movement is complete. The indicator [11] is a marker that becomes visible, or sits in a more visible position, once said afore movement(s) is/are complete to indicate that said microneedles [4] have penetrated the skin and/or been detached. In the embodiment shown, the indicator [11] sits within the housing [5a] and, upon depression of the housing [5a], or a part thereof, indicator [11] becomes visible, typically but not exclusively, via a corresponding window in the housing [5a] that accommodates the indicator [11].

Referring to FIG. 3, there is shown an exemplar microneedle for use in the device. FIG. 3A shows a side view of the microneedle [4], which includes a neck region [4a] or indent that forms a point of weakness. As will be appreciated, such a point of weakness facilitates breakage of same upon lateral movement and thus detachment of the needle [4] from the carrier [3] for embedding/insertion in the skin. In this arrangement, it has been advantageously found that the detached microneedles exhibit improved anchorage in the skin. Preferably, the point of weakness is manufactured at or towards the base of the microneedle so that said microneedles are delivered into the skin. Further, as shown by the perspective views in FIGS. 3B, the microneedles (4) are semi-circular in cross-section. In this arrangement, preferably, the microneedles [4] are aligned so that their curved surfaces face away from the downward stroke of the actuator [6] and their flat surfaces face towards the downward stroke of the actuator [6], thereby during lateral movement the greatest force is applied to the weakest region of the microneedle neck to ensure the breakage of same. This is best illustrated with reference to FIG. 4.

Further, preferably the microneedles [4] are dissolvable/degradable or substantially dissolvable/degradable so that they have the ability to pass or dissolve into the surrounding environment, such as when in contact with an aqueous environment or the skin. As will be appreciated by those skilled in the art, dissolvable/degradable microneedles can deliver any agent coated thereon or therein into the skin. Accordingly, ideally, said microneedles [4] comprise at least one therapeutic, pharmaceutical or biological agent for delivery into the skin and release into the body. Said agent(s) include(s) active agent intended for topical, local, and/or systemic delivery. Generally, any drug or agent which can be delivered intradermally can be delivered using the microneedles. The agent includes any conventional medicament, therapeutic, contraceptive or cosmetic agent, vaccine, protein, antibody, or structural agent. Thus, microneedles [4] which are detached from the device and left embedded in the skin can provide sustained or extended release of agent(s) being delivered by the microneedles [4].

FIG. 5 shows a cross-sectional view of the device during various stages of its operation. Cap 12 is removed and the device [1] is placed against a site to be injected, in particular the underside of the device is placed against the skin. Movement of said second housing [5a], by the depression of same (or a part of same) with respect to housing [2a], generates a downward stroke which, via actuators [6] contacting one of carriers [3], plates [7] or movable supports [8], results in carriers [3] and/or plates [7] extending to a position where the microneedles [4] project beyond said first housing [2a] and into skin (FIG. 5B). Moreover, continued downward movement of said second housing [5a] is translated, by a stopping member [10] that prevents further downward movement of support [8], into a lateral force as actuator [6] slides between the upper part of said carriers [3] or plates [7] thus forcing them laterally along the movable support [8] (FIG. 5C) and resulting in a shearing force being exerted on said inserted microneedles [4] which thus break at their point of weakness (FIG. 5D). At this point the microneedles [4] are detached from said housing [2a] and remain in the skin. Thus, a single downward depression results in microneedles [4] being extended and inserted into adjacent skin and then sheared away from the housing [2a].

Typically, the device [1] is made from materials that are disposable thus the device is a single disposable unit that, once positioned, and the upper part is depressed, microneedles are inserted and detached in a single stroke. If present, an indicator signals the completion of the down stroke, and so also lateral stroke that occurs as a consequence. The applicator can then be disposed of, ideally after replacing the cap.

The invention therefore helps to solve the problems associated with agent or medicament delivery in a simple fashion that can be used by trained and even inexperienced staff, and advantageously can be used at home or in the field by a patient.

1. A device for introducing microneedles into the skin of a subject said device comprising:
a first part comprising at least one carrier having integral or associated therewith a plurality of microneedles wherein said carrier is moveably mounted in a first housing to move with respect to said housing from a retracted to an extended position where, when in said extended position, said microneedles or part thereof project beyond said housing and further wherein said at least one carrier is also moveably mounted in said housing to move with respect to said housing laterally or at 90° with respect to said movement that produces the afore extension; and
a second part comprising at least one actuator which is mounted in, or integral with, a second housing that is adapted to engage with said first housing, wherein said actuator and/or said second housing is movable with respect to said first housing so that said actuator is functionally coupled with said at least one carrier whereby, when the actuator is activated:
i) the actuator exerts a force to move said carrier with respect to said first housing from said retracted to said extended position and, when in said extended position,
ii) said actuator exerts a force to move on said carrier with respect to said first housing laterally or at 90° with respect to said movement that produces the afore extension, thereby breaking the microneedles to leave parts of same in the skin.

2. The device according to claim 1 wherein said at least one carrier is mounted on or in a plate or between a pair of plates.

3. The device according to claim 1 wherein a plurality of said carriers are mounted on or in a plurality of plates or between pairs of plates.

4. The device according to claim 3 wherein a plurality of said carriers or plates are mounted on said movable support.

5. The device according to claim 1 wherein said carrier(s) or plate(s) is/are mounted on a movable support to move with respect to said first housing from said retracted to an extended position.

6. The device according to claim 1 wherein said carrier(s) or plate(s) is/are mounted on a movable support in said first housing to move with respect to said first housing laterally, or at 90° with respect to said movement that produces the said extension.

7. The device according to claim 3 wherein movement of said movable support brings about synchronised movement of said carriers or plates whereby said carriers or plates move from said retracted to an extended position in a synchronised fashion.

8. The device according to claim 3 wherein a plurality of said movable supports are provided, each carrying a plurality of said carriers or plates.

9. The device according to claim 1 wherein said actuator makes contact with at least one of said carrier(s) or plate(s) or movable support(s).

10. The device according to claim 9 wherein said actuator, when activated, is adapted to produce a downward stroke that moves said carriers or plates from said retracted to said extended position.

11. The device according to claim 9 wherein said actuator, when activated, is adapted to produce a downward stroke that moves said carrier(s) or plate(s) laterally or at 90° with respect to said movement that produces the said extension.

12. The device according to claim 10 wherein said actuator, when activated, is adapted to produce a downward stroke that after, moving said carriers or plates from said retracted to said extended position, moves said carrier(s) or plate(s) laterally or at 90° with respect to said movement that produces the said extension.

13. The device according to claim 1 wherein said device comprises a stopping member for limiting the amount of downward motion of at least one of said carrier(s) or plate(s) or movable member(s).

14. The device according to claim 3 wherein during said lateral movement a number of said carriers or plates move laterally in a first direction and another number move laterally in a second opposite direction, with respect to said first direction.

15. The device according to claim 14 wherein about an equal number of carriers or plates move in said first and second directions.

16. The device according to claim 1 wherein at least one of said housings comprises an indicator which indicates when the said downward and/or extended and/or lateral movement is complete.

17. The device according to claim 16 wherein said indicator is a marker that becomes visible, or sits in a more visible position, once said afore movement(s) is/are complete.

18. The device according to claim 1 wherein said microneedle(s) are tapered and further include a neck region or indented region which forms a point of weakness for the breakage of same.

19. The device according to claim 1 wherein said microneedles are semi-circular in cross section and aligned so that, in use, their flat surface faces towards the actuator and their curved surface away from said actuator.

20. The device according to claim 1 wherein said microneedles are dissolvable or degradable or substantially dissolvable or degradable.

21. The device according to claim 1 wherein said microneedles comprise at least one therapeutic, pharmaceutical, cosmetic, or biological agent for delivery into the skin.

22. The device according to claim 1 comprising a removable shield or cap adapted to prevent the relative movement of the first part or first housing and second part or second housing.

23. The device according to claim 1 wherein said actuator is manually operated.

24. A method for inserting microneedles into skin of a subject, said method comprising the steps of:
a) applying to the skin of said subject a device comprising a first part comprising at least one carrier having integral or associated therewith a plurality of microneedles wherein said carrier is moveably mounted in a first housing to move with respect to said housing from a retracted to an extended position where, when in said extended position, said microneedles or part thereof project beyond said housing and further wherein said at least one carrier is also moveably mounted in said housing to move with respect to said housing laterally or at 90° with respect to said movement that produces the afore extension;
a second part comprising at least one actuator which is mounted in, or integral with, a second housing that is adapted to engage with said first housing, wherein said actuator and/or said second housing is movable with respect to said first housing so that said actuator is functionally coupled with said at least one carrier whereby, when the actuator is activated:
i) the actuator exerts a force to move said carrier with respect to said first housing from said retracted to said extended position and, when in said extended position,
ii) said actuator exerts a force to move on said carrier with respect to said first housing laterally or at 90° with respect to said movement that produces the afore extension, thereby breaking the microneedles to leave parts of same in the skin;
b) activating the actuator of the device to move the at least one carrier or at least one plate with respect to said first housing from a retracted to an extended position to insert the microneedles into the skin;
c) further activating or continuing activation of said actuator so that said actuator exerts a force on said at least one carrier or at least one plate to move it/them with respect to said first housing laterally, or at 90° with respect to said movement that produces the afore extension, to detach the microneedles from the carrier or plate; and
d) removing the device from the skin.
